(19) United States
(12) Patent Application Publication
(10) Pub. No.: US 2001/0053533 A1
(43) Pub. Date: Dec. 20, 2001

(54) Prion-binding activity in serum and plasma

(76) Inventors: Adriano Aguzzi, Zurich (CH); Michael Boris Fischer, Dubendorf (CH)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(21) Appl. No.: 09/899,407

(22) Filed: Jul. 5, 2001

Related U.S. Application Data
(63) Continuation of application No. 09/407,667, filed on Sep. 28, 1999.

(51) Int. Cl. G01N 33/53; G01N 33/542; G01N 33/553

(52) U.S. Cl. 435/7.9; 436/526

(57) ABSTRACT

Disclosed are methods and tools for the concentration and detection as well as quantification of pathological prion proteins as well as agents to be used in said detection and/or in the prevention or treatment of prion diseases. Said agents are factors with prion binding activities found in blood serum and blood plasma.

### FIELD OF THE INVENTION

The present invention concerns a method and agents to detect transmissible spongiform encephalopathies as well as agents for the prevention and treatment of respective infections.

### BACKGROUND ART

According to all available evidence, the agents causing transmissible spongiform encephalopathies, termed prions, are devoid of informational nucleic acids and consist of an “infectious” protein (termed PrP^{Sc}) capable of converting a normal host protein called prP^{C} into a likeness of themselves. The only organ system in which histopathological damage and its clinical sequelae can be demonstrated as a consequence of infection with prions is the nervous system (Brandner et al., 1996). This consideration applies to both the human transmissible spongiform encephalopathies, such as Creutzfeldt-Jakob disease, Gerstmann-Straussler-Scheinker Syndrome, Kuru and fatal familial insomnia, and all known prion encephalopathies of animals (Weber and Aguzzi, 1997). The latter comprise scrapie in sheep, bovine spongiform encephalopathy, and chronic wasting diseases of mule, deer and exotic ungulates (Weissmann and Aguzzi, 1997).

However, there is no doubt that prions, herewith operationally defined as the infectious agents causing transmissible spongiform encephalopathies, can colonize organs other than the central and peripheral nervous system, and can be demonstrated in extracerebral compartments (Aguzzi et al., 1997). The problem of which organ systems can harbour infectivity is further complicated by the existence of prion strains. Just like strains of conventional viruses, prions can come in various different flavors, each one of which has its specific preferences with regard to the host range which is infectible and also to the type of cells in which it replicates (Aguzzi, 1998). One paradoxical situation, which is of immediate relevance to the question of blood safety, is exemplified by the radically different organ tropism of the BSE agent in cows and in humans. BSE prions seem to be largely confined to the neural compartment of cows, even after oral exposure (Wells et al., 1998). A very accurate study of the pathogenesis of experimental BSE in cows upon feeding 100 grams of infected brain has disclosed that there is only a short and transient period during which infectivity can be demonstrated in the terminal ileum (Wells et al., 1998). At later time points, BSE prions can only be shown in brain, spinal cord, and dorsal root ganglia. The exact localization of BSE in the terminal ileum is not known. It is being discussed whether infectivity resides in Peyer's patches or in the neural compartment which comprises the Plexus submucosus Meissner and the Plexus myentericus Auerbach. There is a great body of circumstantial evidence that BSE prions can provoke new variant Creutzfeldt-Jakob Disease (nvCJD) (Bruce et al., 1997; Chazot et al., 1996; Hill et al., 1997; Will et al., 1996), but no absolutely final evidence has been produced. For the purpose of the following discussion we will regard the evidence that BSE and new variant Creutzfeldt-Jakob Disease caused by the same agent as sufficiently verified (Aguzzi and Weissmann, 1996). Upon passage into humans, and consecutive progression to manifest nvCJD, prions experience a dramatic shift in their organotropism. Instead of remaining confined mainly to neural structures, they can be detected in many organs belonging to the immune system, including most notably tonsils, spleen, and as recently demonstrated, the appendix (Hilton et al., 1998). It is, therefore, unavoidable to conclude that the tropism of the infectious agent for various structures depends on both the strains of prions in question (and therefore it is in part autonomous to its carrier) and on the species in which prion disease manifests itself (Aguzzi and Weissmann, 1998).

These considerations are not only of academic interest. In fact, the transmissibility of the agent by iatrogenic manipulations (i.e. blood transfusions, organ transplants, etc.) is crucially affected by such parameters.

### Horizontal Transmissibility of Human Prions

Prion diseases of humans are undoubtedly transmissible. However, transmission is achieved only under particular circumstances. One could say that in this respect prion diseases fulfill the characteristics of transmissibility delineated by Semmelweiss for puerperal fever: these affections are infectious but not contagious. Direct transmissions of brain-derived material from a patient suffering from Creutzfeldt-Jakob disease to other persons have documentedly resulted in transmission of disease. A particularly tragic case occurred in the early seventies in Zurich, when electrodes used for cortical recordings from Creutzfeldt-Jakob patients were sterilized (formaldehyde and alcohol) and used in additional patients. Disease was transmitted to the very young recipients (Bernoulli et al., 1977). Also, transplantation of cornea has most likely resulted in transmission of disease (Duffy et al., 1974).

Despite these tragic dimensions, cases of iatrogenic transmission of CJD via neurosurgical procedures have remained rather rare. This is not totally understood, given that the frequency of subclinical CJD must be much higher than that of manifest disease, and that most neurosurgical instruments are not sterilized in a way that would reliably inactivate prions. Therefore, the quite rare nature of iatrogenic transmission is likely to indicate that host factors, in addition to the virulence of prions, may affect the probability that infection takes place. This notion is strengthened by the epidemiology of iatrogenic CJD (iCJD) upon transmission of contaminated dura mater. It has been estimated that several thousands patients, predominantly in Japan, may have been exposed to the CJD agent via preparations of cadaveric dura mater which had been contaminated with prions. However, it appears that less then 2% of those exposed have developed disease so far. While we can rejoice about this low efficiency in the “take” of infectivity, we do not fully understand the biological basis for the apparent protection enjoyed by most subjects exposed to CJD prions. The largest problem with iatrogenic transmission has occurred as result of administration of pituitary hormones of cadaveric origin (Gibbs et al., 1985). Preparations of growth hormone and of gonadotropins contaminated with human prions have caused the death of more then 80 persons, predominantly children. Due to the long latency that can be expected when the agent is introduced into extracerebral sites, such as via intramuscular injection, it must be assumed that further cases from this procedure, which has been stopped more than a decade ago, will arise in the future.

Besides its tragic human dimension and the harm that it has cost to the patients and to their physicians, the pituitary hormone disaster needs to be understood in detail, because the anterior lobe of the pituitary gland is not a part of the central nervous system. Therefore, these events may serve as a paradigm for transmission of prions via contaminated extracerebral tissue that does not belong to the canonical sites of replication of prions. The observation that latency after intracerebral contamination is much shorter than latency after peripheral infection is in good agreement with experimental data from various animal models, and suggest that a rather lengthy phase of extracerebral events (which may include replication of the agent, and invasion of specific extraneuronal systems) may be a precondition to prion neuroinvasion (Aguzzi, 1997).

### Factors Influencing the Neurotropism of Prions

There is good reason to suspect that neuroinvasive processes in the course of prion infections are very tightly controlled. Perhaps the best argument in this respect derives from the observation that the incubation times of experimental animals inoculated intraperitoneally with scrapie prions are extremely reproducible. Upon inoculation with a known amount of standard inoculum, the experience in various laboratories has been that latencies between inoculation and first clinical symptoms display standard variations in the order of only a few percent points (Klein et al., 1997). If prion neuroinvasion were a totally random process, one would expect a large variability in the incubation times, which would depend on processes governed by chance. However, if some rate-limiting processes control neuroinvasion, these may be responsible for the remarkable precision of the incubation times. Indeed, we very much hope that this interpretation is correct because if such processes exist they might be amenable to manipulation, which in turn may represent a post-exposure strategy to prevent overt prion disease. Indeed, various mechanisms have been explored by which neuroinvasions may be accomplished.

A first phase or neuroinvasion seems to be widespread colonization of the immune system. This colonization can be visualized by homogenizing spleen, lymph nodes, tonsils, and also appendix, and injecting the homogenates into suitable experimental animals. The dilution of the homogenates at which 50% of the experimental animals become sick, contains one ID50 of the infectious agent in each inoculum.

The second phase of neuroinvasion seems to be dependent upon a compartment which cannot be replaced by adoptive bone marrow transfer (Blättler et al., 1997) and which may be represented by the peripheral nervous system and/or the follicular dendritic cells resistant to germinal center of secondary lymphatic organs. It appears that this second compartment necessitates the expression of normal prion protein in order to support neuroinvasion (Blättler et al., 1997).

Neuroinvasion is dependent on a functional immune system, and immunodeficient mice do not develop disease after inoculation with a moderate dose of the agent (Fraser et al., 1996; Kitamoto et al., 1991; Lasmezas et al., 1996; O'Rourke et al., 1994). One crucial component of the immune system necessary for neuroinvasion has been traced to the physical presence of terminally mature B-lymphocytes . To date, it is not clear whether B cells are required because they bind physically prions and carry them to sites of neuroinvasion, or whether B cells produce factors, or induce processes, which are indirectly responsible for facilitating neuroinvasion (Klein et al., 1997). Given the requirement for B-lymphocytes secreting lymphotoxin for the maturation of follicular dendritic cells, and the fact that follicular dendritic cells accumulate large amounts of scrapie prions in experimental situations, it is tempting to speculate that the main function of B-lymphocytes in the aforementioned process consists in allowing FDCs to mature.

### The Cellular and Molecular Basis of Prion Neuroinvasion

Following experimental inoculation of mice with prions at peripheral sites, there is typically a prolonged, clinically silent replication phase of the infectious agent within the lymphoreticular system (LRS). This occurs prior to detectable neuroinvasion by prions and the subsequent occurrence of neurological symptoms. During this preclinical latency period, prions may replicate to high titers within lymphoreticular tissues. Elucidating the cell types in which prions replicate within the peripheral lymphoid tissue and—crucially—how prions are transported to the central nervous system (CNS) is of great interest and clinical importance. Despite considerable evidence implicating the role of the immune system in peripheral prion pathogenesis, there have been few studies on the identity of the cells involved in this process. It has been shown many years ago that whole-body irradiation of mice with gamma rays fails to influence prion pathogenesis or incubation time of scrapie. This has been taken as an argument against significant involvement of proliferating cells in the lymphoreticular phase of prion propagation. Instead, follicular dendritic cells (FDC) have been considered as the prime cell type for prion replication within lymphoid tissue since PrP^{Sc} accumulates in the follicular dendritic network of scrapie infected wild-type and nude mice (Kitamoto et al., 1991). In addition, severe combined immuno deficient mice (SCID), which lack mature B- and T-cells, and which do not appear to have functional FDCs, are highly resistant to scrapie after intraperitoneal inoculation and fail to replicate prions in the spleen (Fraser et al., 1996; Kitamoto et al., 1991; Lasmezas et al., 1996; O'Rourke et al., 1994). Interestingly, bone-marrow reconstitution of SCID mice with wild-type spleen cells restores full susceptibility to scrapie after peripheral infection (Fraser et al., 1996; Klein et al., 1998). These findings suggest that an intact, or at least partially functional, immune system comprising lymphocytes and FDC is required for efficient transfer of prions from the site of peripheral infection to the CNS.

The time course for the development of scrapie disease following intracerebral or intraperitoneal inoculation is highly reproducible and is primarily dependent on the dose of the inoculum. Therefore, neuroinvasion by prions migrating from peripheral lymphoid tissue may depend on tightly controlled, rate-limiting reactions. In order to identify such rate-limiting steps during prion neuroinvasion, PrPC deficient mice bearing PrP-overexpressing cerebral neurografts were infected intraperitoneally (i.p.). No disease was observed in the grafts, suggesting that neuroinvasion depends on PrP expression in extracerebral sites. This was further underlined by reconstitution of the lymphoid system with PrPC expressing cells, which restores infectivity in the lymphoid tissue, but still fails to transport prions to the nervous system.

As prions can be detected in lymphoreticular tissues, an understanding of the peripheral pathogenesis is of immediate importance in assessing risks of iatrogenic transmission of human BSE via exposure to blood or tissues from preclinical cases, and possibly from contaminated surgical instruments, or even blood and blood products. Additionally, such advances might pave the way for the development of sensitive diagnostic tests and the means to block prion neuroinvasion. Why is contamination of the blood supply with prions an important issue? The main problem is new variant CJD. For one thing, we by far do not know as much about the epidemiology and iatrogenic transmissibility of this new disease as we do for sporadic CJD (sCJD). What is most unsettling, the distribution of preclinical disease in Great Britain and possibly in other countries is very obscure, and the little knowledge that is being gathered is far from reassuring (Will et al., 1999). Moreover, there is all reason to believe that nvCJD may be much more “lymphoinvasive” than its sporadic counterpart. In particular, nvCJD prions can be easily detected in lymphatic organs such as tonsils and appendix (Hill et al., 1999; Hill et al., 1997; Hilton et al., 1998), a fact that was previously demonstrated to be true for scrapie (Schreuder et al., 1997; Schreuder et al., 1998; Vankeulen et al., 1996), but not for sCJD prions. While all available evidence points to follicular dendritic cells as the prion reservoir in lymphatic organs, splenic lymphocytes of experimentally inoculated mice can be infected with prions (Raeber et al., 1999). Although prion infectivity of circulating lymphocytes appear to be at least two logs lower than that detected in splenic lymphocytes (Raeber et al., 1999), the possibility that circulating lymphocytes may be in equilibrium with their splenic siblings call for cautionary measures. The nature of the latter is still matter of controversy and debate: leukodepletion has been advocated, but at present there is no certainty about its efficacy, and even whether the presently available technologies for leukoreduction are necessary and/or sufficient for decreasing the threat to blood supply that derives from nvCJD. In addition, it has to be taken into account that, even if blood prion infectivity were to be originally contained in lymphocytes in vivo, lysis of cells may lead to contamination of non-particulate fractions and, in the absence of appropriate measures of removal, of stable blood products.

The second consideration applies so secondary prophylaxis. Given the very large numbers of infectious BSE material that has entered the human food chain, it is possible that many individuals harbor preclinical nvCJD. It is imperative and urgent to develop strategies that will help control spread of the agent and that will hopefully prevent the clinical outbreak of symptoms in these persons. Possible targets for the interference with neuroinvasion are rate-limiting processes that control prion replication within the infected individual. In light of the knowledge discussed above, treatments that target the neuro-immune interface of prion replication and neuroinvasion (Aguzzi and Collinge, 1997) seem a promising area for research aimed at post-exposure prophylaxis.

### Methods to Detect Prions and Their Limitation

In the age of real-time kinetic polymerase chain reaction (PCR), we have become very spoiled with respect to the detection thresholds which we demand from assays geared at detecting viral contaminants in blood. Consider the case of HIV: here the introduction of quantitative PCR technologies has pushed the limit of detection in blood and blood products down to quasiperfection. Even when PCR techniques have not proved that useful, or have not yet met with such widespread acceptance, ultrasensitive immunochemical methods, such as time-resolved fluorescent ELISA, have progressed to a degree of sophistication that is highly satisfactory for most screening application. So why do we still have a problem with prion detection in blood?

The most formidable problem derives from the unique biology of the prion. According to more-or-less accepted wisdom, infectious prions are likely to consist solely of the PrP^{Sc} protein, which has exactly the same amino acid structure as the normal cellular protein PrP^{C}. A more noncommittal way of wording this fact would be to state that PrP^{Sc} is the only known surrogate marker for prion infectivity: this latter statement is likely to be agreeable upon by both the proponents of the protein-only hypothesis and by those who still believe that the infectious agent is a virus.

The consequence of the fact mentioned above for prion detection is obvious: if prion-specific nucleic acids do not exist, any PCR-based screening assay to detect said nucleic acid will not be an option. Therefore, we are left with immunochemical assays. Besides being less sensitive than PCR by several orders of magnitude, these are also fraught with a series of prion-specific problems. The biggest trouble, again, derives directly from the peculiar biology of TSE agents. As explained above, PrP^{Sc} possesses the same chemical composition as PrP^{C}, and the latter is a membrane-bound protein that is normally found in many cell types of healthy individuals including white blood cells (Aguzzi and Weissmann, 1997). Although PrP^{C} and PrP^{Sc} differ in a number of physical properties, it appears to be extremely difficult to develop immunological reagents which reliably differentiate between these two isoforms. Only one monoclonal antibody has been described to react with PrP^{Sc} but not with PrP^{C}, and its practical usefulness remains to be demonstrated since fourteen months after its publication no follow-up studies have appeared and even the company which developed this reagent in the first place does not appear to use it in its in-house screening assay for BSE prions.

The hitherto best method for the detection of prions is by performing Western blot analysis with homogenized brain tissue that has been digested with proteinase K (PK). The digestion is necessary since for Western blot analysis the secondary structure is broken up so that no difference is found any more between cellular prions (PrP^{C}) and pathological prions (PrP^{Sc}), however, while PrP^{C} is readily digested by PK under specified conditions, PrP^{Sc} is only degraded to relatively large fragments called PrP²⁷⁻³⁰.

It is also already known to concentrate proteins by adsorbing them to so called magnetic beads (MB) to which a specific antibody is bound. However, the application of such a concentration method to PrP has been assumed to be impossible due to the specific features of prions.

Thus, still a great need exists to have a sensitive method or test to detect small amounts of prions not only for diagnosis but also for further investigating the disease, as well as agents to perform such tests.

### Brief Description of the Invention

Hence it is one object of the present invention to provide a method for the detection of the pathological prion protein as PrP^{Sc} or PrP²⁷⁻³⁰, respectively.

It is another object of the present invention to provide a method for searching for prion interacting agents.

Still another object of the present invention are agents recognizing PrP^{Sc} and/or PrP²⁷⁻³⁰.

Still another object of the present invention are solid phase materials such as e.g. magnetic beads carrying such agents and composition comprising same.

Still another object of the present invention are compositions comprising such agents for purifying body fluids and sterilization of surgical and diagnostic instruments.

In the method of the present invention for the concentration of PrP^{Sc} or digestion products thereof, a body fluid, such as e.g. blood, urine, cerebrospinal fluid etc., or fluidized organ, such as brain tissue, lymph nodes, tonsils etc., is treated with a solid phase material such as magnetic beads(MB) whereby at least part of said material or beads, respectively, carries a prion binding site. A preferred prion binding site is a factor with prion binding activity (PrPB).

The method works very well with a fluidized organ, in particular homogenized tissue of central nervous system, preferably homogenized brain tissue.

In several cases the prion binding site can only distinguish PrP^{C} and PrP^{Sc} in digested form. For such cases, it is necessary to digest the fluid or fluidized organ prior to the actual concentration step. A suitable digestion is obtained by digestion with proteinase K (PK), whereby it is important to inactivate the proteinase K prior to the addition of the solid phase, e.g. MBs.

Preferred solid phase materials carrying PrPB are prepared by coupling such materials with blood serum, or blood plasma, such as fresh frozen plasma of mammals. Even more preferred solid materials carrying PrPB are prepared by treating solid material with serum or plasma fraction II of ammonium sulfate precipitation, or with plasma fraction I of ammonium sulfate precipitation (see below). Most preferred solid materials carrying PrPB are prepared by treating MBs with factors found in specific fractions and designated PrPBIp or PrPBIIp or PrPBIIs (see below).

Very suitable solid materials are magnetic beads since they can easily be treated with specific components of interests and easily be collected by applying a magnetic field.

A further method of the present invention concerns the detection (and optionally quantification) of PrP^{Sc} or digestion products thereof, wherein PrP^{Sc} is first concentrated as described above, optionally also with previous digestion of the fluid or fluidized organ, and then detected and optionally compared with a standard. A suitable detection method is Western blot analysis. Such test may furthermore be embodied by other detection methods such as a microtiter plate format immunoassay (e.g. ELISA assay), an immunoprecipitation assay, a BIACORE assay, immunocytochemical assay, histoblot assay etc.

Besides of the above mentioned methods, the present invention also concerns factors with prion binding activities such as PrPBIIs, which is a prion binding activity in fraction II of ammonium sulfate precipitation of serum, PrPBIIp, which is a factor with prion binding activity in fraction II of ammonium sulfate precipitation of plasma, and PrPBIp, which is a factor with prion binding activity in fraction I of ammonium sulfate precipitation of plasma. Said factors are of course subject matter of the present invention in any form, such as in isolated form, or as ingredient in a composition, e.g. in a fraction of ammonium sulfate precipitation.

Said factors can be obtained by concentration and/or isolation of PrPBs whereby serum or plasma is subjected to fractionated ammonium sulfate precipitation thus that a PrPB of interest is precipitated, preferably in only one fraction. A further purification can be obtained by the application of further protein isolation methods.

The factors of the present invention are not only suitable for the detection of prions, but they have further applications in methods for the purification and removal of pathological prion protein from body fluids and organs, such as blood, urine, cerebrospinal fluid, brain tissue, lymph nodes, tonsils etc., or for the sterilization of surgical and/or diagnostic tools, basing on the affinity of PrPB for the pathological prion protein. They are furthermore tools for a therapy regimen based on the modulation of production of PrPB for preventing the spread of prions in the body. Especially suitable in this respect is PrPBIp, that is also especially suitable for the purification of body fluids, e.g. blood units. Such purification may e.g. be performed by treating fluids with PrPBIp, in particular with immobilized PrPBIp or plasma fractions containing same.

Also part of the present invention is a test for the detection of pathological prion protein in body fluids or organs such as blood, urine, cerebrospinal fluid, brain tissue, lymph nodes, tonsils etc, that utilizes the specific binding properties of PrPB to pathological prion protein. Such test can be embodied as a microtiter plate format immunoassay, e.g. ELISA assay, an immunoprecipitation assay, a BIACORE assay, immunogytochemical assay, histoblot assay etc.

Also the DNA sequences specific for biosynthesis of PrPB are comprised by the present invention as well as vectors able to express such DNA sequences in suitable hosts.

Furthermore comprised by the present invention are: a method for purification of PrPB by using PrP²⁷⁻³⁰ as bait; monoclonal and polyclonal antibodies produced in animals such as mice, rabbits, chicken etc., and directed against PrPB; single-chain Fv fragments and other types of fragments of antibodies produced in recombinant phages or in other recombinant systems, and directed against PrPB; a test predictive of susceptibility to prion diseases based on polymorphisms of PrPB, or on variations in the strength and pattern of production of PrPB; a transgenic animal, e.g. mouse that overproduction PrPB in brain, lymph nodes, or other organs, to be used in a bioassay for prions; a knockout animal, in particular a mouse, which is devoid of PrPB, to be used in a bioassay for prions; a production method of PrPB by expressing a DNA sequence specific for the biosynthesis of PrPB in a suitable host cell, such as bacteria, yeast, fungi, or eukaryotic cells, and by purification of PrPB from the aforementioned organisms; a use of natural or synthetic, preferably purified PrPB as a medicament for therapeutical applications in humans and animals; a vaccination of organisms with natural or synthetic PrPB, in particular PrPBIp; a diagnostic assay for human and/or animal diseases resulting from abnormal production and/or metabolism of PrPB.

### Brief Description of the Drawings

FIG. 1 is a scheme showing the IAP method.

FIG. 2 shows Western Blots and IAP experiments of dilution experiments, whereby lanes 1 to 6 and 10 represent usual Western Blots and lanes 7 to 9 and 11 to 13 represent immuno affinity purification (IAP).

FIG. 3 is a scheme showing the prion affinity assay (PAA) method.

FIG. 4 represents Western Blots showing serum related observations with normal mouse serum.

FIG. 5 represents Western Blots showing the results with Ig-deficient and C3-deficient material.

FIG. 6 represents Western Blots showing the results with Ig and IgG.

FIG. 7 represents Western Blots showing PAA with normal mouse serum proteins and serum proteins of B cell deficient mice.

FIG. 8 represents Western Blots showing PAA with serum proteins of PrP-deficient mice.

FIG. 9 represents Western Blots showing PAA with bovine serum albumin.

FIG. 10 represents Western Blots showing pH dependency.

FIG. 11 represents Western Blots showing PAA of ammonium sulfate precipitates.

FIG. 12 represents Western Blots showing PAA using sheep antibodies preincubated with different ammonium sulfate precipitates.

FIG. 13 represents Western Blots showing PAA using different ammonium sulfate precipitates of human plasma.

### DETAILED DESCRIPTION OF THE INVENTION

As already mentioned above, there is a great need for a detection method for low concentrations of PrP^{Sc} that can be used as a diagnostic test for transmissible spongiform encephalopathies (TSEs).

There are basically three diagnostic principles for TSEs: histopathological detection of the typical spongiform changes in the CNS, detection of the scrapiespecific isoform of the prion protein, and the bioassay that detects infectivity. All these methods have limitations: histopathology is not useful for preclinical diagnosis since the structural changes appear late in the incubation period. Detection of the scrapie the Western specific is form of prion protein is more sensitive but still much less sensitive than the bioassay. The bioassay can, in principle, detect as little as 1 infectious unit but can last months or even years.

The hitherto used Western blot technique is based on the partial protease resistance of PrP^{Sc} that allows to distinguish between PrP^{C} and PrP^{Sc}. After protease treatment, PrP²⁷⁻³⁰—the protease resistant core of PrP^{Sc}—can be detected but not PrP^{C} which is completely digested.

Although due to the “stickiness” of prions it was generally assumed that immuno affinity purification (IAP) cannot be applied, it has now been found that concentration can be achieved by applying magnetic beads (MB) carrying a prion binding site, preferably a factor with prion protein binding activity (PrPB).

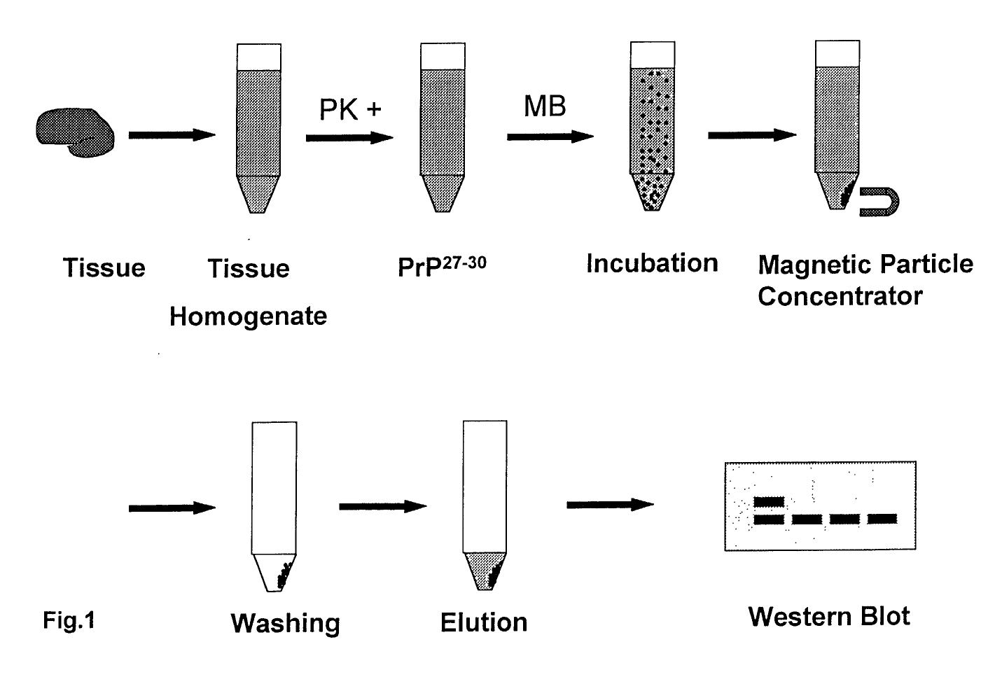

Thus, because the sensitivity of detection of absolute amounts of PrP²⁷⁻³⁰ is a function of antibody affinity, and cannot be easily increased for each given antibody, in the scope of the present invention, despite of the hitherto assumed problems, first an “immuno affinity purification” (IAP) assay has been developed, using antibodies covalently crosslinked to solid phase material, e.g. magnetic beads. Because the monoclonal antibody (6H4 purchased at Prionics, Zurich, Switzerland, described in Korth et al., 1997), originally used for the development of the IAP, is not able to distinguish between PrP^{C} and PrP^{Sc} (it binds both undigested forms as well as digested PrP^{Sc}, i.e. PrP²⁷⁻³⁰), it is necessary to perform Proteinase K digestion prior to the IAP (see FIG. 1).

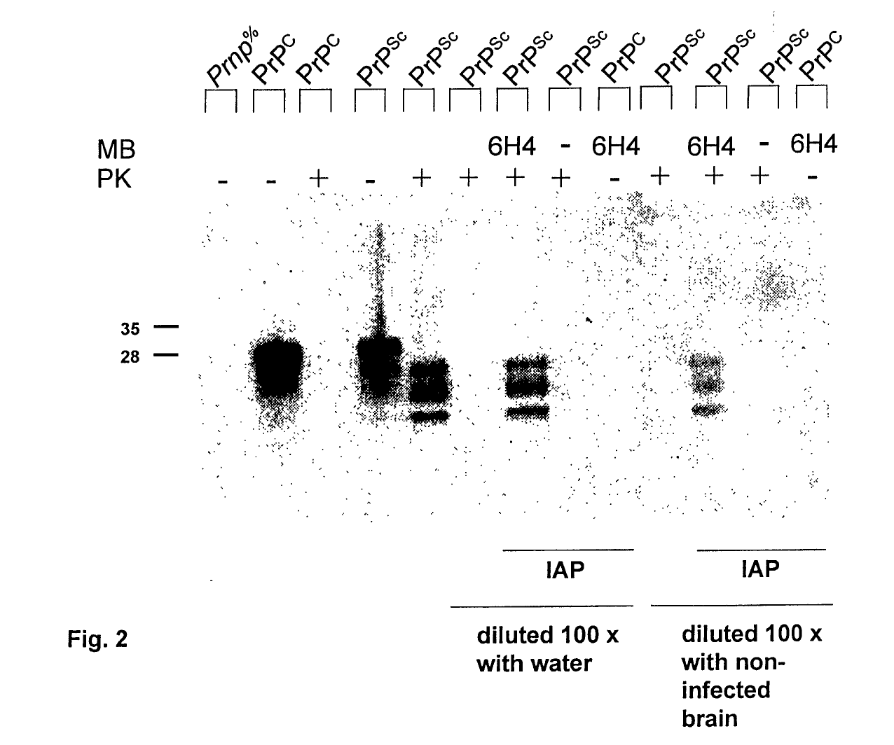

For the development of the present IAP method, the following model system was used: Two tests were performed to determine the efficiency of the method. On the one hand, small amounts of a scrapie-infected mouse brain homogenate were diluted with water and then subjected to the PrPSC concentration method. On the other hand, small amounts of a scrapie-infected brain homogenate was diluted with brain homogenate of non-infected mice in order to simulate a real situation in which a brain homogenate contains low amounts of PrP^{Sc} (see FIG. 2).

In FIG. 2, lanes 1 to 6 and 10 represent usual Western Blots and lanes 7 to 9 and 11 to 13 represent immuno affinity purification (IAO). PrnP % is material from PrP deficient mice. MB are of course only used for IAP whereby 6H4 refers to MB coupled with 6H4 antibodies and—refers to uncoupled MBs. PRP^{C} refers to brain homogenate of non-infected mice and PrP^{Sc} refers to brain homogenate of scrapie-infected mice. PK refers to Proteinase K digestion whereby—refers to non digestion and+to digested homogenate. The same abbreviations are used for the following figures.

For prion analysis in homogenate, in particular of brain tissue, it is important to use in a first homogenation step low concentration of ionic detergent, followed by low speed centrifugation, preferably 500 g 30 minutes, 4° C. applied twice. For following steps high concentration of non-ionic detergent is used and a protein concentration of the homogenate of at most 5 mg/ml.

Conditions for the proteinase K digestion are preferably 50 μg/ml PK, 37° C. and at least half on hour.

Suitable incubation conditions for the beads with homogenate are e.g. about 1.5 hours at room temperature, whereby for low concentrations longer incubation times might be preferable.

The concentration step in said first attempt was carried out by adding to digested homogenate magnetic beads (MB) carrying said 6H4.

If a digestion step is needed, it has to be performed prior to the concentration step, whereby the digestion, usually by proteinase K, has to be stopped prior to the concentration step by deactivating the proteinase e.g. with phenyl methyl sulfonyl fluoride or another agent known to the skilled person.

By applying the method of the present invention for e.g. brain tissue homogenate, PrP²⁷⁻³⁰ can be concentrated up to amounts detectable by Western blot analysis from tissue comprising much less pathological prion protein than needed for the hitherto known tests.

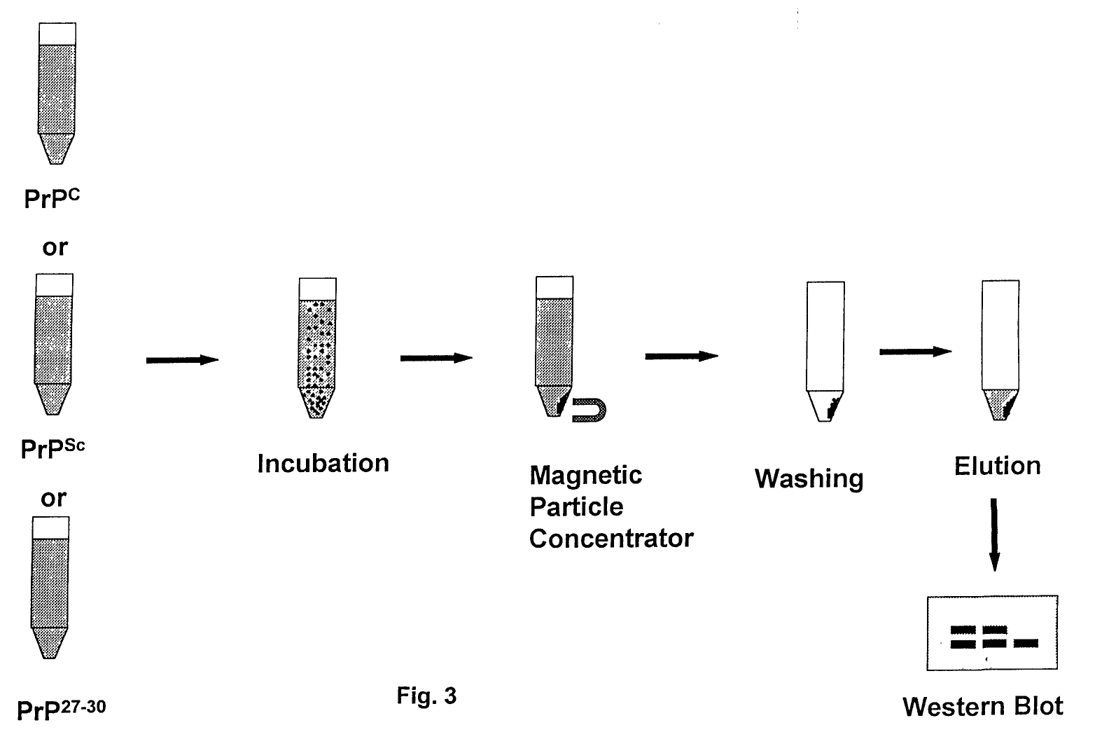

Using largely the same procedure, the above described method can also be applied as prion affinity assay (PAA) by exchanging the monoclonal antibody 6H4 by other substances to be examined, for example in order to find a binding partner for PrP^{Sc} (see FIG. 3).

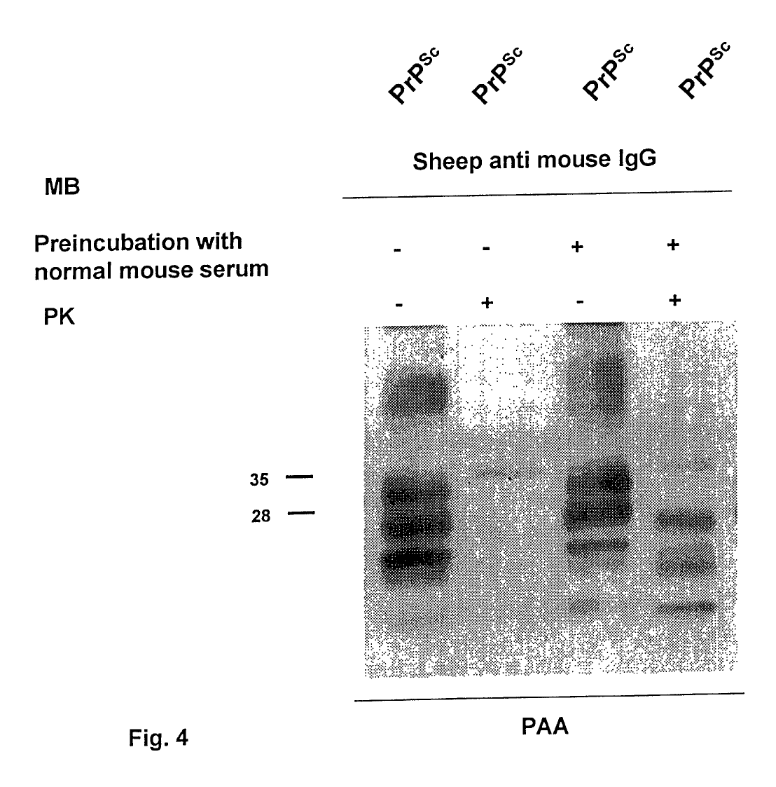

Using this assay it could furthermore be shown that in the concentration and detection method of the present invention MB coupled with blood serum or blood plasma (in the following referred to as carrying serum or plasma) are also suitable agents. Thereby different anchoring methods can be applied for fixing serum or plasma on solid carriers, e.g. MBs, such as direct anchoring or anchoring on an antibody such as immunoglobulin. Even by using raw serum and plasma, with pathological prion protein effects as with 6H4 have been obtained. No activity was found with non-digested PrP^{C}. I is likely that a large variety of sera and plasma can be used, since it was found that e.g. MBs coupled with sera of sheep immunized with mouse immunoglobuilns are also active (see FIG. 4).

This effect could even be enhanced if the serum or plasma was fractionated. A wide- spread method for such fractioning is precipitation with ammonium sulfate.

If such precipitation is performed with concentrations of 0-25% (fraction I), 25-50% (fraction II), 50-75% (fraction III) and 75-100% (fraction IV), all prion protein binding activity for serum is found in fraction II. A further fractioning showed that a larger amount of activity is present in a 25-33% fraction than in a 33-50% fraction.

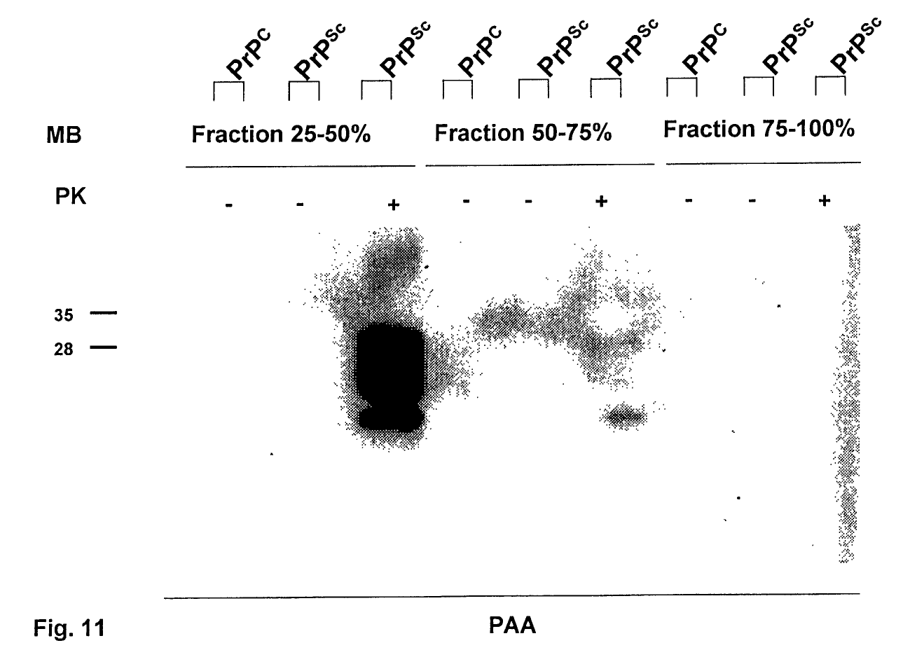

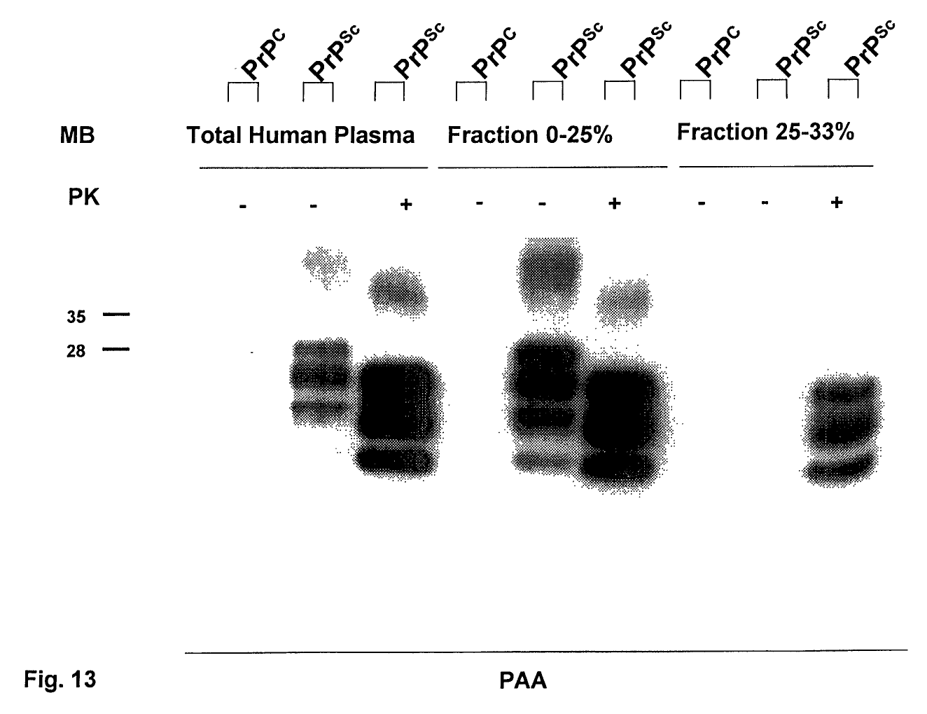

While whole serum and whole plasma, as well as their fractions II recognize PrP^{Sc} in digested form, i.e. as PrP²⁷⁻³⁰, it has furthermore be found that plasma is also able to bind non digested PrP^{Sc}. A further investigation showed that the activity able to recognize undigested PrP^{Sc} is—upon fractioning—found in fraction I while the activity only recognizing the digested form is found in fraction II (see FIGS. 11 and 13, abbreviations see FIG. 2).

The prion protein binding activity is furtheron designated PrPB, whereas the activity selectively recognizing the digested PrP^{Sc}, i.e. PrP²⁷⁻³⁰ is furtheron called PrPBIIs or PrPBIIp with II for fraction II, s for serum and p for plasma, while the activity recognizing undigested PrP^{Sc} is furtheron referred to as PrPBI or PrPBIp.

By performing tests with sera and plasma of different animal and human origin, very good evolutionary conservation of the activity was found since all tested sera and plasma from mouse to human did recognize the pathological protein.

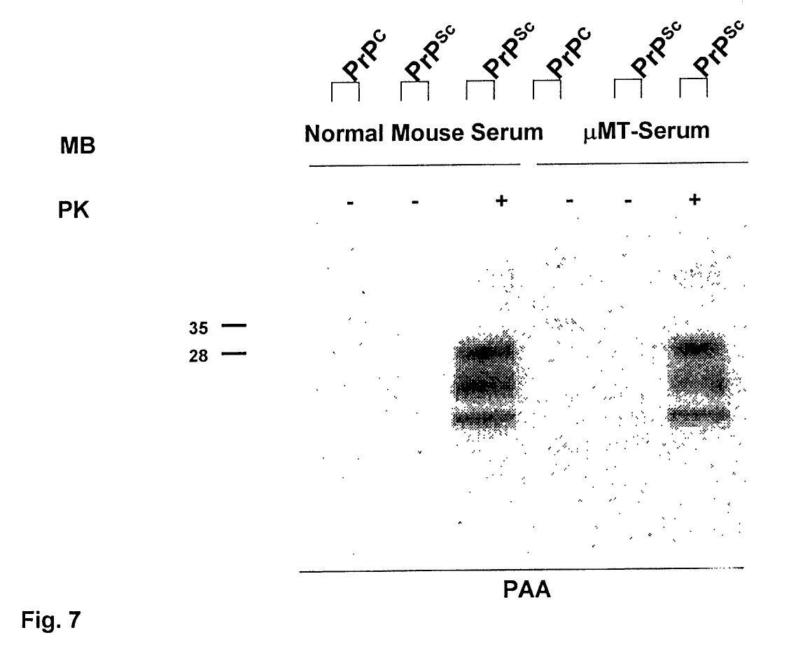

It could thus be shown that PrPBIIs and PrPBIIp do not bind the normal form of the cellular prion protein PrP^{C}; instead they bind PrP²⁷⁻³⁰ (the proteinase-K-treated form of the pathological prion protein PrP^{Sc}) (see FIGS. 7, 11, 13). Therefore, PrPBII is acting as a specific reagent that recognizes exclusively pathologically folded, infectious prion protein after pretreatment of the sample, yet does not recognize the normal noninfectious counterpart. In contrast thereto, PrPBIp also recognizes non-digested PrP^{Sc}.

Here serum factors are described, that are called “PrPB” (Prion protein binding activity). Said factors are present in serum or plasma of animals such as humans and mice. Furthermore developed was an affinity assay that is called the “PAA (prion affinity assay)” and that allows to identify PrPB in organic fluids or fluidized organs (FIG. 3). Using this assay, it could be shown that different sera, such as mouse serum and human serum, are capable of binding PrP²⁷⁻³⁰ with an extremely high capacity and with an affinity that is similar to that displayed by the monoclonal antibody 6H4 (Korth et al., 1997). This is one of the best anti-PrP antibodies commercially available.

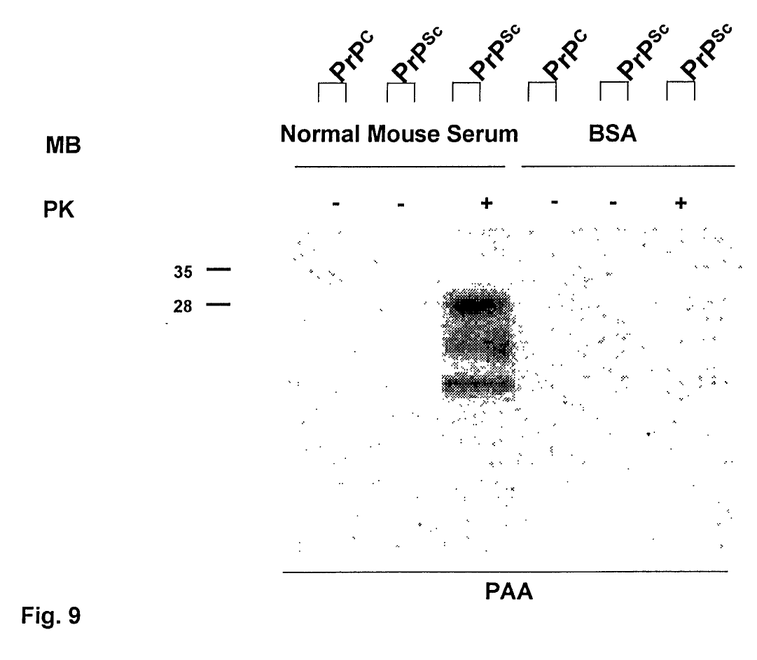

It could be shown that PrPBII is not an immunoglobulin, because it is present in the serum of B-cell deficient μMT mice which do not contain any immunoglobulins (these mice are agammaglobulinemic) (see FIG. 7), and because purified mouse immunoglobulins do not react with PrP²⁷⁻³⁰. Finally, PrPBII is not albumin, since purified albumin does not immunoprecipitate PrP²⁷⁻³⁰ (see FIG. 9, lanes 4 to 6, BSA=bovine serum albumin).

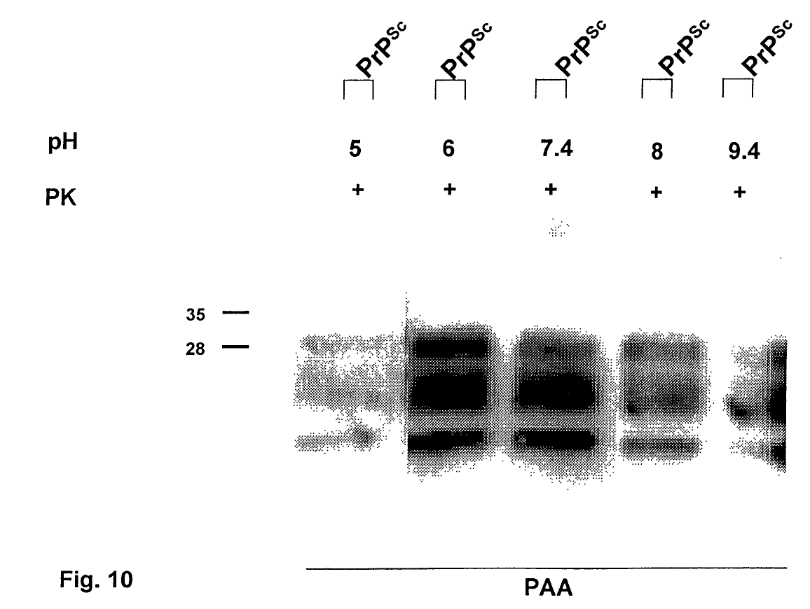

PrPBII binds to PrP27-30 at an optimum of pH 6 to 7.4, and binding is abruptly decreased at lower or higher values (see FIG. 10).

When blood is fractionated using ammonium sulfate precipitation, PrPBII partitions with the precipitate at concentrations of 25-50% (see FIG. 11) and to a larger amount at concentrations of 25-33%. This is a further line of evidence that reinforces that PrPBII is not an immunoglobulin, and also excludes a large number of other candidate molecules.

### EXAMPLES

### Example 1

### IAP Method

The IAP protocol is the following: Bring the brain tissue in a 15 ml FALCON tube, put it on ice and leave it there for all steps. Add Homogenate Buffer (0.5% DOC/0.5% NP-40 in PBS) to get 10% (w/v) homogenate. Pass the tissue through a 18 gauge needle and a 22 gauge needle by sucking up and down for 15 times each. Centrifuge the homogenate for 30 minutes at 500 g and 4° C. Keep the supernatant. Determine the protein concentration. Centrifuge the homogenate for 30 minutes at 500 g and 4° C. Keep the supernatant. If the protein concentration is higher than 10 mg/ml then bring the homogenate to a protein concentration of 10 mg/ml using the homogenate buffer. Bring the homogenate to a protein concentration of 5 mg/ml and 3% Tween 20/3% NP-40 all in PBS. Add to the tissue homogenate Proteinase K to get a final concentration of 50 μg/ml. Incubate for 60 minutes at 37° C. Add PMSF to get a final concentration of 5 mM. Add 0.25 volumes of IAP buffer (3% Tween 20/3% NP-40 in PBS). Resuspend the magnetic beads (covered with 6H4) according to the protocol described below) thoroughly. Pipette out 100 μl. Remove buffer. Add the homogenate to the beads and incubate the bead-sample mixture with continous mixing for 1.5 hours at room temperature. Collect the beads using the MPC (strong magnet). Wash three times with 1 ml Washing Buffer (2% Tween 20/2% NP-40 in PBS) and once with 1 ml PBS by vortexing for 15 seconds at room temperature and by using the MPC. Spin down the beads, discard the remaining supernatant using again the MPC. Add 24 μl×Loading Buffer (50 mM Tris pH 6,8; 2% SDS; 0.01% bromphenol blue; 10% glycerol) . Heat to 95° C. for 5 minutes. If the samples are stored at −20° C. then heat them again for 30 seconds at 95° C. before performing SDS-PAGE followed by western Blot: Assemble the glass plates according to the manufacturer's instructions. Prepare in a Falcon tube the appropriate volume of the Resolving Gel (2.1 ml H₂O, 1.5 ml 40% Acrylamid, 1.3 ml 1.5 M Tris pH 8.8, 50 μl 10% SDS, 50 μl 10% Ammoniumpersulfat, 2 μl TEMED). Mix the components in the order shown. Polymerization will begin as soon as the TEMED has been added. Pour the acrylamide solution into the gap between the glass plates. Leave sufficient space for the stacking gel (the length of the comb plus 1 cm). Using a pasteur pipette carefully overlay the acrylamide with water. Place the gel in a vertical position at room temperature. After polymerization is complete (30 minutes), pour off the overlay and wash the top of the gel several times with deionized water to remove any unpolymerized acrylamide. Prepare in a Falcon tube the appropriate volume of the Stacking Gel (1.48 ml H₂O, 0.25 ml 40% Acrylamid, 0.25 ml 1.0 M Tris pH 6.8, 20 μl 10% SDS, 20 μl 10% Am-moniumpersulfat, 2 μl TEMED). Mix the components in the order shown. Polymerization will begin as soon as the TEMED has been added. Pour the stacking gel solution directly onto the surface of the polymerized resolving gel. Immediately insert a clean Teflon comb into the stacking gel solution, being careful to avoid trapping air bubbles. Place the gel in a vertical position at room temperature. After polymerization is complete (30 minutes), remove the Teflon comb carefully. Mount the gel in the electrophoresis apparatus. Add Running buffer to the top and bottom reservoirs. Remove any (25 mM Tris, 250 mM glycine, 0.1% SDS) bubbles that become trapped at the bottom of the gel between the glass plates. Load 24 μl of each of the samples in a predetermined order into the bottom of the wells (1. well: Low -range marker). Load an equal volume of 1×Gel-loading Buffer into any wells that are unused. Attach the electrophoresis apparatus to an electric power supply (the positive electrode should be connected to the bottom reservoir). Apply 10 V/cm to the gel. After the dye front has moved into the resolving gel (30 minutes), increase the voltage to 14 V/cm and run the gel until the bromophenol blue reaches the bottom of the resolving gel (1 hour). Then turn off the power supply. Cut six sheets of absorbent paper (Whatman 3MM or equivalent) and one sheet of nitrocellulose to the size of the gel (6 cm×8 cm). If the paper overlaps the edge of the gel, the current will short-circuit the transfer and bypass the gel, preventing efficient transfer. Wet the absorbent paper, the nitrocellulose and the gel by soaking in Transfer (39 mM glycine, 48 mM Tris, 0.037% SDS, 20% methanol) Buffer. On the bottom plate of the apparatus (the anode), assemble the gel, nitrocellulose, and paper in this order:

bottom electrode,

three layers absorbent paper soaked in transfer buffer,

one nitrocellulose membrane soaked in transfer buffer,

polyacrylamide gel slightly wetted with transfer buffer,

three layers absorbent paper soaked in transfer buffer.

Check carefully for air bubbles and gently remove them either by using a gloved hand or by rolling a pipet over the sandwich. Dry any buffer that may surround the gel-paper sandwich. Carefully place the upper electrode (the cathode) on top of the stack. Put a weight on it. Connect the electrodes and commence transfer. Running time is 1 hour with a current of 1 mA/cm². After transfer, disconnect the power source. Carefully disassemble the apparatus. Mark membrane to follow orientation (usually by snipping off lower left-hand corner, the number one lane). Rinse the membrane three times with TBS-T. Add Blocking Buffer (5% (w/v) nonfat dry milk in TBS-T). Incubate at room temperature with agitation for 30 minutes. Rinse the membrane three times with TBS-T. Add to 2.5 μl of mAB 6H4 (2 mg/ml) 12.5 ml of 1% (w/v) nonfat dry milk in TBS-T. Incubate at room temperature with agitation for 1 hour or overnight at 4° C. Remove the membrane from the antibody solution and wash three times for 10 minutes each in TBS-T. Add to 1.25 μl of relativ anti mouce IgGlHRP 12.5 ml of 1% (w/v) nonfat dry milk in TBS-T. Incubate at room temperature with agitation for 1 hour. Remove the membrane from the antibody solution and wash three times for 15 minutes each in TBS-T. Mix 1 ml of detection solution 1 with 1 ml of detection solution 2 from the ECL Western blotting detection reagents (Amersham Pharmacia Biotech). Incubate for precisely 1 minute at room temperature without agitation. Drain off excess detection reagent by putting the membrane on a absorbent paper. Gently place the membrane, protein side down, on a SaranWrap. Close SaranWrap to form a envelope avoiding pressure on the membrane. Place the membrane, protein side up, in the film cassette. Work as quickly as possible. Switch off the lights and carefully place a sheet of autoradiography film such as (Hyperfilm ECL) on top of the membrane, close the cassette and expose for some seconds (15″, 30″).

### Example 2

### PAA Method

Couple the protein of interest to magnetic beads: Bring 100 μg of protein into approx. 1 ml of Coupling Buffer (0.1 M borate buffer pH 9.5: dissolve 6.183 g H3BO3 in 800 ml distilled water, Adjust pH to 9.5 using 5 M NaOH and adjust volume to 1000 ml with distilled water; if necessary, change buffer by dialysis). Make a homogeneous suspension of the Dynabeads M-280 Tosylactivated by Dynal using a pipette and by vortexing for approximately 1 min. Pipette out 1 ml of Dynabeads and wash as follows: Place the tube in the DYNAL MPC. Leave to separate for 2 minutes. Remove the supernatant taking care not to disturb the Dynabeads. Remove the tube from the Dynal MPC and resuspend the Dynabeads in PBS. Repeat these steps and resuspend the Dynabeads in the coupling buffer containing the antibodies. Incubate for 24 h at 37° C. with tilt rotation. Place the tube in the magnet for 3 minutes and remove the supernatant. Wash the coated Dynabeads six times: 2× in PBS/BSA (add 0.1% (w/v) bovine serum albumin (final concentration) to PBS), pH 7.4 for 5 minutes at room temperature; 1× in Blocking Buffer (0.2 M Tris pH 8.5 with 0.1% (w/v) BSA: dissolve 2.42 g Tris in 80 ml distilled water. Adjust pH to 8.5 using 1 M HCl, add 0.1% BSA and adjust volume to 100 ml with distilled water) for 4 h at 37° C.; 1×0 in PBS/BSA, pH 7.4 for 5 minutes at room temperature; 1× in 1% Tween 20 for 10 minutes; 1× in PBS/BSA, pH 7.4 for 5 minutes at room temperature. Store the coated Dynabeads in PBS/BSA pH 7.4, 0.02% sodium azide. Then prepare Sample I: Add 1 ml of PAA Buffer (3% NP-40/3% Tween 20 in PBS) to 10 μl of not infected brain homogenate (Protein concentration 5 mg/ml; 0.5% DOC/0.5 NP-40). Then prepare Sample II and III: Add 1 ml of PAA Buffer (3% NP-40/3% Tween 20 in PBS) to 10 μl of infected brain homogenate (Protein concentration 5 mg/ml; 0.5% DOC/0.5 NP-40). Incubate Sample I and Sample II for 30 minutes at 37° C. without PK. Incubate Sample III for 30 minutes at 37° C. with PK at final concentration of 50 μg/ml (add 50 μl of PK 1 mg/ml). Add PMSF to all samples to get a final concentration of 5 mM (add 50 μl of 100 mM PMSF). Resuspend the Magnetic Beads thoroughly. Pipette out 100 μl. Add the beads to the Samples and incubate the bead-sample mixture with continuous mixing for 1.5 hours at room temperature. Collect the beads using the MPC. Wash three times with 1 ml Washing Buffer and once with 1 ml PBS by vortexing for 15 seconds at room temperature and by using the MPC. Spin down the beads, discard the remaining supernatant using again the MPC. Add 24 μl 1× Loading Buffer. Heat to 95° C. for 5 minutes. If the samples are stored at −200° C. then heat them again for 30 seconds at 95° C. before loading on the gel.

### Example 3

The PAA experiment of Example 2 was repeated using magnetic beads covered with sheep anti mouse IgG that was pre-incubated with normal mouse serum. PrP^{Sc} and PrP²⁷⁻³⁰ were recognized (see FIG. 4).

### Example 4

In order to further investigate the type of molecule of PrPB, the procedure of Example 2 was carried out with specific sera, etc.

Since first results were obtained with specially treated sheep antibodies covered MBs, it was tested whether said antibody themselves recognize something that is associated with PrP^{Sc} or possess themselves an affinity to PrP^{Sc}, or do they recognize in the serum anything that interacts with PrP²⁷⁻³⁰.

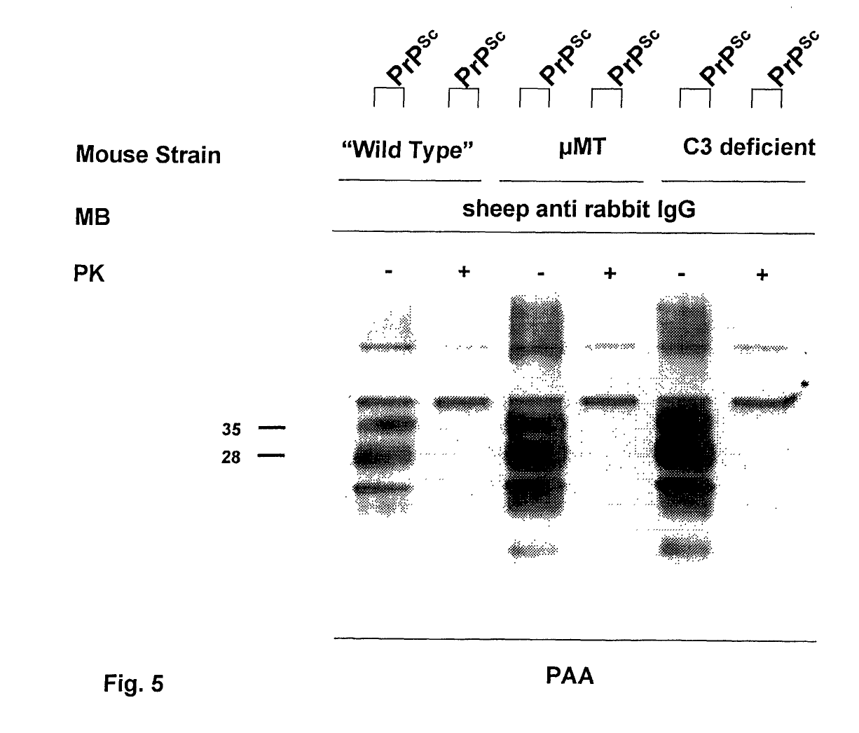

For this purpose the beads were incubated with scrapie material of B-cell deficient mice which do not contain any immunoglobulins (these mice are agammaglobulinemic). Still PrP^{Sc} was recognized. So it is not immunoglobulines associated with PrP^{Sc} that are recognized by the beads. The same experiment was performed with scrapie material of mice which do not contain the complement factor and again PrP^{Sc} was recognized. In addition, it could also be excluded that the beads recognize C3 (see FIG. 5).

### Example 5

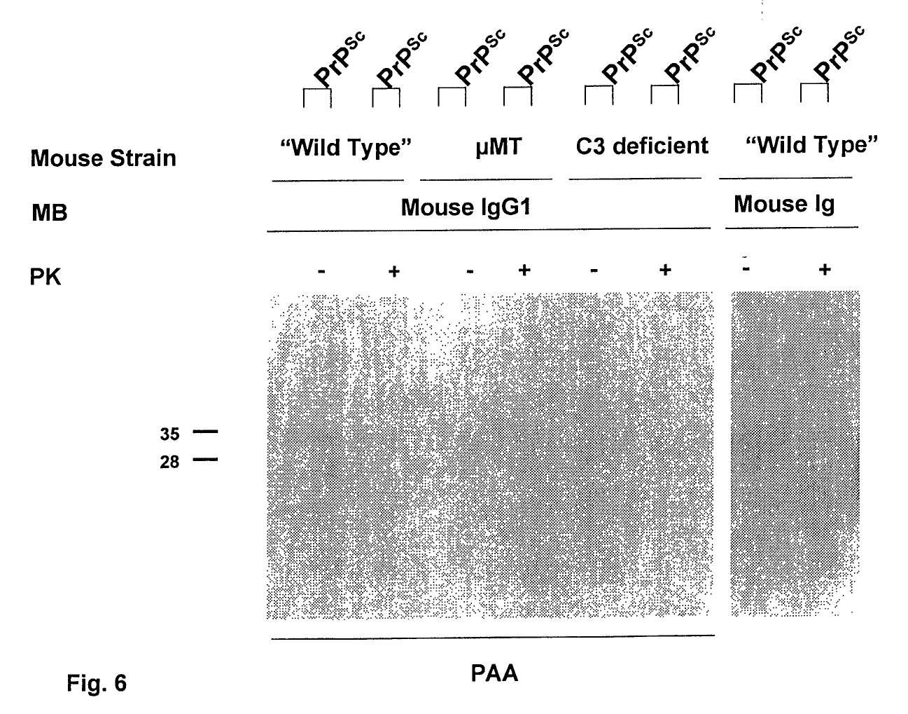

It was furthermore investigated whether Ig themselves recognize in a non specific way PrP^{Sc}. Therefore IgG and total Ig fraction were coupled to beads but no affinity to Prp^{Sc} or Prp²⁷⁻³⁰ could be detected (see FIG. 6).

### Example 6

In a further test, total serum proteins were coupled to magnetic beads and the PAA was repeated. Both examined sera, mouse serum and human serum, are capable of binding PrP²⁷⁻³⁰ with an extremely high capacity and with an affinity that is similar to that displayed by the monoclonal antibody 6H4 (Korth et al., 1997), one of the best anti-PrP antibodies commercially available.

### Example 7

Investigations were performed to obtain information on the physical nature of the PrPB. By applying the method of Example 2 it could be shown that PrPB is not an immunoglobulin, because it is present in the serum of B-cell deficient mice (see FIG. 7).

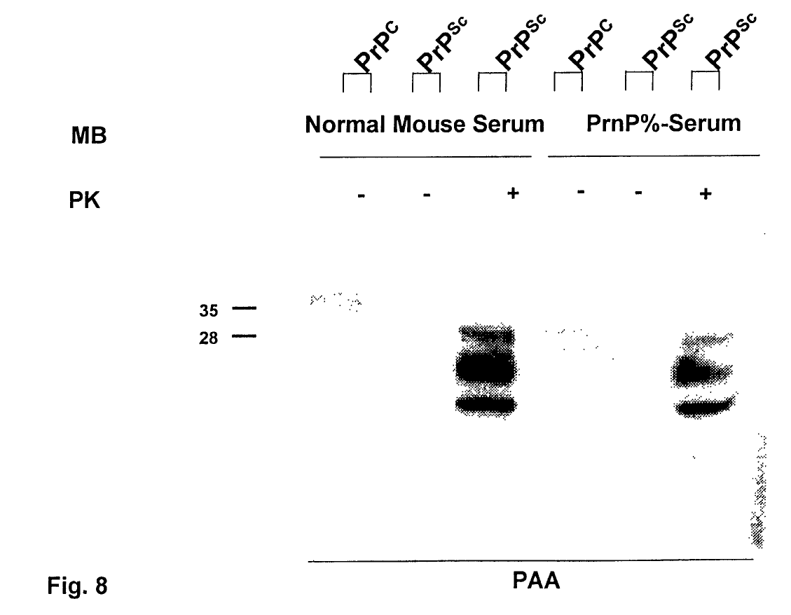

It is also not identical with PrP^{C} as it is also present in the serum of PrP % mice. This also indicates that PrPB is produced in the absence of PrP^{C} (see FIG. 8).

Finally, PrPB is not albumin, since purified bovine albumin does not show affinity to PrP²⁷⁻³⁰ (see FIG. 9).

### Example 8

The method of Example 2 was performed under different pH conditions. Therewith it could be shown that the PrPB binds to PrP²⁷⁻³⁰ at an optimum of around pH 7, and binding is abruptly decreased at lower or higher values (see FIG. 10).

### Example 9

When mouse or human serum is fractionated using ammonium sulfate precipitation and used for the method of Example 2, the results show that PrPBIIs is found in the precipitate at concentrations of 25-50% (see FIG. 11).

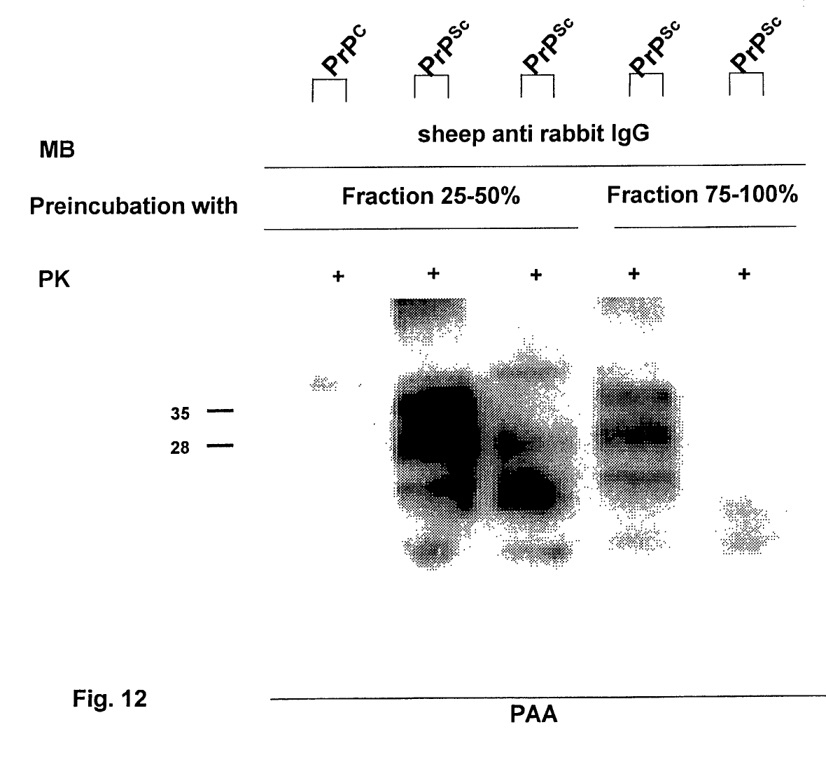

Another very important finding is the fact that the magnetic beads covered with the sheep antibodies (purchased from Dynal, Oslo, N-0212, Norway) do recognize something in the positive ammonium sulfate fraction that contains the PrPB but not in the fractions that do not contain the PrPB (see FIG. 12).

### Example 10

By incubating the beads with serum of B cell deficient mice and performing a silver stain of the SDS-PAGE of the eluate and using them in the method of Example 2 showed that the sheep antibodies recognize something else than IgG (no Figure).

### Example 11

Further investigations with human plasma (instead of human serum), performed as described in Example 2, revealed even a second binding factor (PrPBIp). Total human plasma proteins do not bind to PrP^{C} but to non digested PrP^{Sc} and to PrP²⁷⁻³⁰. The PrP^{Sc} affinity is only present in the ammonium sulfate precipitate at concentrations of 0-25%. In the precipitate at concentrations of 25-33% remains only the affinity for PrP²⁷⁻³⁰ as it is also found in serum (see Examples above). Results obtained with serum are shown in FIG. 13.

While there are shown and described presently preferred embodiments of the invention, it is to be distinctly understood that the invention is not limited thereto but may be otherwise variously embodied and practiced within the scope of the following claims.

### References

Aguzzi, A. (1997). Neuro-immune connection in spread of prions in the body? The Lancet 349, 742-743.

Aguzzi, A. (1998). Protein conformation dictates prion strain. Nat Med 4, 1125-6.

Aguzzi, A., Blattler, T., Klein, M A., Raber, A. J., Hegyi, I., Frigg, R., Brandner, S., and Weissmann, C. (1997). Tracking prions: the neurografting approach. Cell Mol Life Sci 53, 485-95.

Aguzzi, A., and Collinge, J. (1997). Post-exposure prophylaxis after accidental prion inoculation. Lancet 350, 1519-20.

Aguzzi, A., and Weissmann, C. (1997). Prion research: the next frontiers. Nature 389, 795-798.

Aguzzi, A., and Weissmann, C. (1998). Spongiform encephalopathies. The prion's perplexing persistence. Nature 392, 763-4.

Aguzzi, A., and Weissmann, C. (1996). Spongiform encephalopathies: a suspicious signature. Nature 383, 666-7.

Bernoulli, C., Siegfried, J., Baumgartner, G., Regli, F., Rabinowicz, T., Gajdusek, D. C., and Gibbs, C. J. (1977). Danger of accidental person-to-person transmission of Creutzfeldt-Jakob disease by surgery [letter]. Lancet 1, 478-479.

Blättler, T., Brandner, S., Raeber, A. J., Klein, M. A., Voigtländer, T., Weissmann, C., and Aguzzi, A. (1997). PrP-expressing tissue required for transfer of scrapie infectivity from spleen to brain. Nature 389, 69-73.

Brandner, S., Isenmann, S., Raeber, A., Fischer, M., Sailer, A., Kobayashi, Y., Marino, S., Weissmann, C., and Aguzzi, A. (1996). Normal host prion protein necessary for scrapie-induced neurotoxicity. Nature 379, 339-43.

Bruce, M. E., Will, R. G., Ironside, J. W., McConnell, I., Drummond, D., Suttie, A., McCardle, L., Chree, A., Hope, J., Birkett, C., Cousens, S., Fraser, H., and Bostock, C. J. (1997). Transmissions to mice indicate that ‘new variant’ CJD is caused by the BSE agent [see comments]. Nature 389, 498-501.

Chazot, G., Broussolle, E., Lapras, C., Blattler, T., Aguzzi, A., and Kopp, N. (1996). New variant of Creutzfeldt-Jakob disease in a 26-year-old French man [letter]. Lancet 347, 1181.

Duffy, P., Wolf, J., Collins, G., DeVoe, A. G., Streeten, B., and Cowen, D. (1974). Possible person-to-person transmission of Creutzfeldt-Jakob disease. N Engl J Med 290, 692-3.

Fraser, H., Brown, K. L., Stewart, K., McConnell, I., McBride, P., and Williams, A. (1996). Replication of Scrapie in Spleens of Scid Mice Follows Reconstitution With Wild-Type Mouse Bone Marrow. Journal of General Virology 77, 1935-1940.

Gibbs, C. J., Jr., Joy, A., Heffner, R., Franko, M., Miyazaki, M., Asher, D. M., Parisi, J. E., Brown, P. W., and Gajdusek, D. C. (1985). Clinical and pathological features and laboratory confirmation of Creutzfeldt-Jakob disease in a recipient of pituitary-derived human growth hormone. N Engl J Med 313, 734-8.

Hill, A. F., Butterworth, R. J., Joiner, S., Jackson, G., Rossor, M. N., Thomas, D. J., Frosh, A., Tolley, N., Bell, J. E., Spencer, M., King, A., AlSarraj, S., Ironside, J. W., Lantos, P. L., and Collinge, J. (1999). Investigation of variant Creutzfeldt-Jakob disease and other human prion diseases with tonsil biopsy samples. Lancet 353, 183-9.

Hill, A. F., Desbruslais, M., Joiner, S., Sidle, K. C., Gowland, I., Collinge, J., Doey, L. J., and Lantos, P. (1997). The same prion strain causes vCJD and BSE [letter] [see comments]. Nature 389, 448-50.

Hill, A. F., Zeidler, M., Ironside, J., and Collinge, J. (1997). Diagnosis of new variant Creutzfeldt-Jakob disease by tonsil biopsy. Lancet 349, 99.

Hilton, D. A., Fathers, E., Edwards, P., Ironside, J. W., and Zajicek, J. (1998). Prion immunoreactivity in appendix before clinical onset of variant Creutzfeldt-Jakob disease [letter]. Lancet 352, 703-4.

Kitamoto, T., Muramoto, T., Mohri, S., Doh ura, K., and Tateishi, J. (1991). Abnormal isoform of prion protein accumulates in follicular dendritic cells in mice with Creutzfeldt-Jakob disease. J. Virol. 65, 6292-6295.

Klein, M. A., Frigg, R., Flechsig, E., Raeber, A. J., Kalinke, U., Bluethmann, H., Bootz, F., Suter, M., Zinkernagel, R. M., and Aguzzi, A. (1997). A crucial role for B cells in neuroinvasive scrapie. Nature 390, 687-90.

Klein, M. A., Frigg, R., Raeber, A. J., Flechsig, E., Hegyi, I., Zinkernagel, R. M., Weissmann, C., and Aguzzi, A. (1998). PrP expression in B lymphocytes is not required for prion neuroinvasion. Nat Med 4, 1429-33.

Korth, C., Stierli, B., Streit, P., Moser, M., Schaller, O., Fischer, R., Schulz-Schaeffer, W., Kretzschmar, H., Raeber, A., Braun, U., Ehrensperger, F., Hornemann, S., Glockshuber, R., Riek, R., Billeter, M., Wuthrich, K., and Oesch, B. (1997). Prion (PrP^{sc})-specific epitope defined by a monoclonal antibody. Nature 390, 74-7.

Lasmezas, C. I., Cesbron, J. Y., Deslys, J. P., Demaimay, R., Adjou, K. T., Rioux, R., Lemaire, C., Locht, C., and Dormont, D. (1996). Immune system-dependent and -independent replication of the scrapie agent. J Virol 70, 1292-5.

O'Rourke, K. I., Huff, T. P., Leathers, C. W., Robinson, M. M., and Gorham, J. R. (1994). SCID mouse spleen does not support scrapie agent replication. J. Gen. Virol. 75, 1511 -1514.

Raeber, A. J., Klein, M. A., Frigg, R., Flechsig, E., Aguzzi, A., and Weissmann, C. (1999). PrP-dependent association of prions with splenic but not circulating lymphocytes of scrapie-infected mice. EMBO J 18, 2702-2706.

Schreuder, B. E., van Keulen, L. J., Smits, M. A., Langeveld, J. P., and Stegeman, J. A. (1997). Control of scrapie eventually possible? Vet Q 19, 105-13.

Schreuder, B. E., van Keulen, L. J., Vromans, M. E., Langeveld, J. P., and Smits, M. A. (1998). Tonsillar biopsy and PrP^{Sc} detection in the preclinical diagnosis of scrapie. Vet Rec 142, 564-8.

Vankeulen, L. J. M., Schreuder, B. E. C., Meleon, R. H., Mooijharkes, G., Vromans, M. E. W., and Langeveld, J. P. M. (1996). Immunohistochemical Detection of Prion Protein in Lymphoid Tissues of Sheep With Natural Scrapie. Journal of Clinical Microbiology 34, 1228-1231.

Weber, T., and Aguzzi, A. (1997). The spectrum of transmissible spongiform encephalopathies. Intervirology 40, 198-212.

Weissmann, C., and Aguzzi, A. (1997). Bovine spongiform encephalopathy and early onset variant Creutzfeldt- Jakob disease. Curr Opin Neurobiol 7, 695-700.

Wells, G. A., Hawkins, S. A., Green, R. B., Austin, A. R., Dexter, I., Spencer, Y. I., Chaplin, M. J., Stack, M. J., and Dawson, M. (1998). Preliminary observations on the pathogenesis of experimental bovine spongiform encephalopathy (BSE): an update. Vet Rec 142, 103-6.

Will, R., Cousens, S., Farrington, C., Smith, P., Knight, R., and Ironside, J. (1999). Deaths from variant Creutzfeldt-Jakob disease. Lancet 353, 9157-9158.

Will, R., Ironside J W, Zeidler M, Cousens S N, Estibeiro K, Alperovitch A, Poser S, Pocchiari M, Hofman A, and Smith (1996). A new variant of Creutzfeldt-Jakob disease in the UK. Lancet 347, 921-925.

1. A method for the concentration of PrPS^{C} or digestion products thereof, wherein a body fluid or fluidized organ is treated with solid phase material such as e.g. magnetic beads (MB) whereby at least part of said material or beads, respectively, carries a prion binding site.

2. The method of claim 1, wherein the prion binding site is a factor with prion binding activity (PrPB).

3. The method of claim 1, wherein the fluidized organ is homogenized tissue of central nervous system.

4. The method of claim 1, wherein the fluidized organ is homogenized brain tissue.

5. The method of claim 1, wherein the fluid is a fluid that has been digested by proteinase K (PK).

6. The method of claim 2, wherein the solid phase materials, e.g. MBs, carrying PrPB are prepared by coupling MBs with blood serum, or blood plasma.

7. The method of claim 1, wherein the solid phase materials, e.g. MBs, carrying PrPB are prepared by coupling solid phase material with serum or plasma fraction II of ammonium sulfate precipitation.

8. The method of claim 1, wherein the solid phase materials, e.g. MBs, carrying PrPB are prepared by coupling MBs with plasma fraction I of ammonium sulfate precipitation.

9. The method of claim 1, wherein the solid phase materials, e.g. MBs, carrying PrPB are prepared by coupling solid phase material with PrPBIp or PrPBIIp or PrPBIIs.

10. A method for the detection, and optionally quantification, of PrP^{Sc} or digestion products thereof, wherein PrP^{Sc} is first concentrated according to one of claims 1 to 6 and then detected, and optionally compared with a standard.

11. The method of claim 10, wherein the detection is performed by Western blot analysis.

12. PrPBIIs which is a factor with prion binding activity in fraction II of ammonium sulfate precipitation of serum.

13. PrPBIIp which is a factor with prion binding activity in fraction II of ammonium sulfate precipitation of normal or fresh frozen plasma.

14. PrPBIp which is a factor with prion binding activity in fraction I of ammonium sulfate precipitation of plasma.

15. Solid phase material, such as MBs, carrying a PrPB.

16. A composition for the purification of body fluids and/or the sterilization of surgical or diagnostic tools comprising a PrPB, preferably coupled to a solid phase material.

17. A method for the sterilization of surgical or diagnostic tools wherein said tools are treated with a PrPB comprising composition, preferably a composition comprising PrPB coupled to a solid phase material.

18. A method for the concentration and/or isolation of PrPBs wherein serum or plasma is subjected to fractionated ammonium sulfate precipitation thus that at least one PrPB of interest is precipitated, preferably in only one fraction.

19. The method of claim 18, wherein the PrPB comprising fraction is further purified by further protein isolation methods.

20. A method for purification and/or removal of pathological prion protein from body fluids or fluidized organs, such as blood, urine, cerebrospinal fluid, brain tissue, lymph nodes, tonsils, or for the sterilization of surgical and/or diagnostic tools basing on the affinity of PrPB for the pathological prion protein.

21. A method for the purification of body fluids, e.g. blood units, wherein the fluid is treated with PrPBIp.

22. A therapy regimen based on the modulation of production of PrPB for preventing the spread of prions in the body.

23. The regimen of claim 19, wherein the PrPB is PrPBIp.

24. A test for the detection of pathological prion protein in body fluids or organs such as blood, urine, cerebrospinal fluid, brain tissue, lymph nodes, tonsils etc, that utilizes the specific binding properties of PrPB to pathological prion protein.

25. The test of claim 24 that is embodied as a microtiter plate format immunoassay, e.g. ELISA assay, an immunoprecipitation assay, a BIACORE assay, a immunocytochemical assay, a histoblot assay, etc.

26. A DNA sequence specific for biosynthesis of PrPB and/or an expression vector comprising same.

27. A method for purification of PrPB by using PrP²⁷⁻³⁰ as bait.

28. Monoclonal and polyclonal antibodies produced in animals, such as mice, rabbits, chicken, or other species, and directed against PrPB.

29. Single-chain Fv fragments and other types of fragments of antibodies produced in recombinant phages or in other recombinant systems, and directed against PrPB.

30. A test predictive of susceptibility to prion diseases based on polymorphisms of PrPB, or on variations in the strength and pattern of production of PrPB.

31. A transgenic animal, in particular mouse that overproduces PrPB in brain, lymph nodes, or other organs, to be used in a bioassay for prions.

32. A knockout animal, in particular a knockout mouse, which is devoid of PrPB, to be used in a bioassay for prions.

33. Production method of PrPB by expressing a DNA sequence specific for the biosynthesis of PrPB in a suitable host cell, such as bacteria, yeast, fungi, or eukaryotic cells, and by purification of PrPB from the aforementioned organisms.

34. Use of natural or synthetic PrPB as a medicament for therapeutical applications in humans and animals.

35. A vaccination of organisms with natural or synthetic PrPB, in particular PrPBIp.

36. A diagnostic assay for human and/or animal diseases resulting from abnormal production and/or metabolism of PrPB.
